(19) United States
(12) Patent Application Publication
(10) Pub. No.: US 2010/0185105 A1
(43) Pub. Date: Jul. 22, 2010

(54) AMBULATORY REMOTE VIGILANCE SYSTEM WITH A PULSE DENOISING, ACTIMETRY AND FALL DETECTION DEVICE

(75) Inventor: Jean-Louis Baldinger, Montgeron (FR)

Correspondence Address: Oliff & Berridge, PLC, P.O. Box 320850, Alexandria, VA, 22320-4850, US

(73) Assignee: Institut National des Telecommunications (INT) Groupe des Ecoles des Telecommunications (GET), Evry (FR)

(21) Appl. No.: 12/531,236

(22) PCT Filed: Mar. 12, 2008
(86) PCT No.: PCT/FR08/50420

§ 371 Date: Mar. 26, 2010

(30) Foreign Application Priority Data
Mar. 12, 2007 (FR) 0753768

(51) Int. Cl. A61B 5/02; A61B 5/11

(52) U.S. Cl. 600/500; 600/595

(57) ABSTRACT

A device for measuring the pulse includes
a pulse sensor of photoplethysmographic type preferably worn in the ear, comprising at least one light source, especially an infrared light source, and a component sensitive to the light emitted by the source, especially a single component, and
a denoising system including
an electronic preconditioning circuit designed to eliminate the slow artifacts of a signal representative of the pulse acquired by the light sensitive component and,
at least one microcontroller designed to process the signal delivered by the electronic preconditioning circuit and eliminate the fast artifacts.

The present invention relates to a method and a device for denoising a pulse sensor.

Because of the aging of the population in Europe, telemedecine, that is to say medicine practiced remotely, is experiencing a growth in development in order to respond to hospital overcrowding and to the desire of the elderly or sick to remain at home. The concept of home telemonitoring has been developed so that patients can continue to live at home while being the subject of medical monitoring.

Application FR 2 829 862 discloses a device for detecting the fall of a person and the presence of vital signals relating to said person.

U.S. Pat. No. 7,175,601 discloses a device for measuring the pulse comprising an optical detector of a beam that has passed through tissues and associated with an accelerometer. The analysis of the optical signal sensed in relation to the signal delivered by the accelerometer makes it possible to eliminate from the pulse signal the artifacts due to the movements of the person wearing the device.

Application EP 1 297 784 discloses a device for measuring the pulse comprising two pulse sensors.

Multisensor devices making it possible to measure various physiological parameters are moreover known. In such devices, pulse sensors are used and are for example placed level with the heart or waist.

While determining the measurements of the pulse, artifacts, nuisance signals or noise, may in error be regarded as beats. A foremost criterion to be considered is the value of the beat/artifact discrimination threshold, that is to say the temporal threshold on the basis of which a signal received is considered to be a useful signal (beat) or noise (artifact). At a maximum pulse frequency of 200 beats by minute (bpm), accepted for the targeted adult population, the minimum interval between two beats is 0.3 seconds. It would thus be possible to choose this duration as beat/artifact discrimination threshold. Nevertheless, such a value of 0.3 seconds does not allow complete removal of the edges of fast artifacts for pulse frequencies of less than 200 bpm, in particular normal pulse frequencies of between 50 and 120 bpm.

Moreover, the raw acquisition of the ambulatory pulse, while the patient is moving around, gives rise to a significant dispersion in the measurements. This dispersion in the measurements renders their utilization more complex and also affects their reliability. This dispersion is explained inter alia by the edge effect appearing at the ends of time intervals disturbed in a continuous manner by the artifacts engendered by the diverse movements of the patient. Beat/artifact discrimination may turn out to be difficult with an approximate scheme.

A requirement therefore exists to benefit from a device that is relatively reliable, lightweight, inexpensive, while being efficacious, making it possible especially to reduce the dispersion in the pulse measurements acquired while ambulatory.

The invention is especially aimed at responding to this requirement.

According to one of its aspects, the subject of the invention is a device for measuring the pulse comprising:
a pulse sensor of photoplethysmographic type preferably worn in the ear, comprising at least one light source, in particular an infrared light source, and a component sensitive to the light emitted by the source, and
a denoising system comprising:
   an electronic preconditioning circuit configured to eliminate the slow artifacts of a signal representative of the pulse acquired by the light sensitive component, and
   at least one microcontroller configured to process the signal delivered by the electronic preconditioning circuit and eliminate the fast artifacts.

By “slow artifact” is meant any artifact whose spectrum does not comprise any component at a frequency higher than 0.5 Hz.

By “fast artifact” is meant any artifact whose spectrum does not comprise any component at a frequency less than 0.5 Hz.

By “ambulatory” or “while ambulatory” is meant any situation during which the patient exhibits physical activity corresponding to a motion of said patient or to a movement of his body.

Such a denoising resulting from an electronic preprocessing of the signal acquired by the pulse sensor as well as an algorithmic post-processing performed by the microcontroller makes it possible to reduce by a factor of 10 for example the dispersion of the pulse measurements acquired while ambulatory with respect to traditional raw acquisition. The error margin in the ambulatory measurements may, by virtue of the invention, be reduced to 5% for example.

The device may comprise just a single pulse sensor.

The device may comprise just a single light source and single component sensitive to the light emitted by the light source.

The microcontroller and the preconditioning circuit being for example situated in one and the same box that can be worn by the patient, the invention makes it possible to benefit from a compact and easy to use device.

The invention may allow improved reliability of acquisition of the pulse while ambulatory, that is to say better consideration, during processing, of uncertainty phenomena.

The light source may comprise one or more light-emitting diodes (LEDs) generating an infrared beam, for example at a wavelength of 950 nm, through a tissue, preferably the patient's ear lobe.

When the light source comprises several LEDs, the latter may constitute just a single light source and be supplied by a single generator alone.

The LED or LEDs can be powered in a discontinuous manner. The extinguishing of the LED or LEDs because of the discontinuous power supply may make it possible to remove additive noise.

By “additive noise” is meant the components of the noise with slow variations, for example caused by a change of position of the patient's head with respect to a main source of ambient light.

The power supply current may be a rectangular current. The duty ratio of this power supply current is advantageously less than 1/10, lying for example between 1/40 and 1/10, being for example equal to 1/20. The choice of such a duty ratio may make it possible to reduce the current consumed to power the LED or LEDs, and this may make it possible to benefit from a highly autonomous device that can keep going for a long period. The device according to the invention may for example keep going for at least one month of continuous use without it being necessary to recharge the battery or exchange the cells.

In order to further reduce the consumption of the device, the power supply of an amplifier connected to the light sensitive component may also be interrupted at the start of the extinction phase of the LED or LEDs and restored slightly before the ignition of the LED or LEDs.

Because of the long period for which it can keep going and of the low dispersion of the pulse measurements, the device is particularly suited to measurement of the pulse while ambulatory.

The powering of the LED or LEDs in a discontinuous manner may further make it possible to increase the signal-to-noise ratio of the signal output by the pulse sensor of photoplethysmographic type.

The light sensitive component may comprise a phototransistor configured to sense the infrared beam that has passed through the patient's ear lobe.

As a variant, the infrared beam that has passed through the ear lobe may be sensed by a photodiode or any other light sensitive component.

The electronic circuit may comprise a chain for filtering the sensed signal. The filtering chain may comprise a stage configured to subtract additive noise from the sensed signal.

The filtering chain may comprise a comb filtering stage, configured to filter the frequencies that are multiples of the frequency of the fundamental of the electrical power supply of the light source. In the case of a 50 Hz power supply, this stage is configured to filter the frequencies that are multiples of 100 Hz. This stage comprises for example two acquisition pathways each comprising a sample-and-hold unit, each of these sample-and-hold units being controlled alternately.

The filtering chain may comprise at least one filter configured to cut off the frequencies outside a spectral window of between 0.1 Hz and 20 Hz, better between 0.5 Hz and 10 Hz.

The electronic preconditioning circuit is preferably entirely analog and may be devoid of analog/digital converter.

An entirely analog preconditioning circuit may make it possible, in the case of subsequent digitization of the signal, to optimize the pulse signal to quantization noise ratio. The electronic preconditioning circuit may comprise a voltage comparator with hysteresis, in particular floating, comparing the signal arising from the filtering chain and a delayed version of this same signal. The delay may lie between 20 ms and 40 ms.

The device for measuring the pulse may comprise just a single electronic preconditioning circuit.

The signal is for example put into a rectangular shape on exit from the electronic preconditioning circuit.

The microcontroller may execute a denoising algorithm.

The subject of the invention is also, according to another of its aspects, a mobile monitoring system for a patient, comprising:
a multisensor terminal comprising a box to be worn by the patient, preferably on the belt, the terminal including the device for denoising the measurement of the pulse such as previously defined, and
a local processing base, for example of PC type, for receiving and processing the information sent by the multisensor terminal. This local base is situated for example in the patient's residence or in a plant room when the mobile monitoring system according to the invention is deployed in an old people's home, with or without medical attention, or else in a care center.

The multisensor terminal may send the information to the local base according to a predefined periodicity, in particular according to a periodicity of thirty seconds, or indeed of fifteen seconds.

The system may comprise means for fixing the box to the belt, for example a buckle, a hook, a fixing of Velcro® type or any other appropriate means.

The local base may be configured to process and merge the information sent by the multisensor terminal in order to optionally produce an alarm.

The local base may be designed to transmit to a medical center or to a nursing practice or to a predefined neighbor a warning message when the pulse sensor is not worn by the patient. In the case where the sensor comprises a clip or any other means of fixing at the level of the ear lobe, such a warning message may for example be transmitted when the fixing means is not worn.

Likewise, a warning message may be transmitted by the local base if the multisensor terminal is not worn on the belt by virtue for example of a micro breaker with which the box of the terminal comes into contact when it is fixed to the belt.

Such a device makes it possible for example to detect whether the box of the terminal has been taken off by the patient.

The local base may also be configured to transmit a warning message when the pulse sensor is not connected to the multisensor terminal, for example if a connection wire is cut or else in case of disconnection, intentional or otherwise.

The multisensor terminal may comprise at least one actimetry sensor chosen from the following list: isotropic movement sensor, inclination sensor and fall impact sensor.

The multisensor terminal may comprise a microcontroller configured to interpret and denoise the information coming from at least one actimetry sensor.

The invention may make it possible to obtain in the course of a given duration measurements relating to the pulse and to the actimetry of the patient, such as for example the orientation of his body with respect to a horizontal axis and a vertical axis.

The fall impact sensor may comprise four arms and each arm may comprise in series an inclination detector and an acceleration sensor.

The multisensor terminal may comprise an emergency call button configured to trigger the sending of information to the local base. These information may be the most recent data, having for example been acquired since the previous send of indications.

The multisensor terminal may comprise an on/off switch comprising two sections, the switch being designed to cut off the power supply to the terminal only after the immediate sending to the local base of the most recent data, accompanied by an cue that the multisensor terminal has been turned off. Subsequent to this immediate sending, a last periodic sending of the data before actual switch-off may still take place, repeating in the data transmitted the cue that the multisensor terminal has been turned off.

The subject of the invention is furthermore, according to another of its aspects, a method for measuring the pulse of a person by means of a device for measuring the pulse comprising:
a pulse sensor, preferably worn in the ear, comprising at least one light source and a component sensitive to the light emitted by the source, and
a denoising system comprising:
   an electronic preconditioning circuit configured to eliminate the slow artifacts of a signal representative of the pulse acquired by the light sensitive component, and
   at least one microcontroller configured to process the signal delivered by the electronic preconditioning circuit and eliminate the fast artifacts.

The pulse of the person may be measured while ambulatory.

The method may comprise a step of preprocessing by the electronic preconditioning circuit in the course of which:
the signal arising from the pulse sensor drives a stage for subtracting the additive components of the noise with slow variations contained in the signal,
the signal is thereafter processed by a filtering stage, in particular a low-pass filtering stage with constant propagation time and with removal of the frequencies which are multiples of the fundamental of the electrical power supply, and
the signal is put into logic form by means of a comparator stage.

The method may comprise a step of algorithmic post-processing by the microcontroller, in the course of which:
a removal of the edge effect is performed by subtracting from the number of beats measured per time interval a predefined number of beats per noise-affected zone, in particular a half-beat per noise-affected zone,
the number of beats corrected during the previous time interval prorata-temporis of the noise-affected time is added to the value obtained,
the temporal noising rate for the interval considered is compared with a reference value, and
the beat/artifact discrimination threshold is adapted.

By “noise-affected time” or “temporal noising rate” is meant the artifact temporal rate counted during a given time interval, for example fifteen seconds.

The beat/artifact discrimination threshold can for example be estimated in a recursive manner.

The reference value of the temporal noising rate is for example between 10 and 30%, being for example equal to 17%.

Should the temporal noising rate be greater than the reference value, a denoising failure message may be emitted.

The subject of the invention is furthermore, according to another of its aspects, a method for measuring the pulse of a person by means of a device for measuring the pulse comprising:
a pulse sensor, preferably worn in the ear, comprising at least one light source and a component sensitive to the light emitted by the source and,
a denoising system comprising:
   an electronic preconditioning circuit configured to eliminate the slow artifacts of a signal representative of the pulse acquired by the light sensitive component,
   at least one microcontroller configured to process the signal delivered by the electronic preconditioning circuit and decrease the fast artifacts,
   a local base configured to substitute a predefined alarm value for the measured pulse value in the case of malfunctioning of the pulse sensor and/or when the latter is not worn by the patient and to send an alarm message.

The subject of the invention is furthermore, according to another of its aspects, a method for detecting the fall of a person by means of a device comprising:
a fall sensor and,
at least one microcontroller configured to process the signal delivered by the fall sensor, in which method the microcontroller modifies the frequency of sampling of the measurements of the inclination sensor when a fall has been detected.

The subject of the invention is furthermore, according to another of its aspects, a method for detecting the fall of a person by means of a device comprising:
a fall sensor,
at least one microcontroller configured to process the signal delivered by the fall sensor, and
a component emitting with predefined periodicity a first signal audible by the patient as long as the fall alarm has not been validated and then, when the alarm has been validated a second signal audible by the patient, different from the first.

Such a component is for example known by the name “buzzer” or “noisemaker”. The first signal corresponds to a beep of duration about equal to 100 ms and emitted with a periodicity of between one and ten seconds, for example three seconds and the second signal corresponds to a beep of duration about equal to a second.

The invention may make it possible, by the emission of the second signal, to warn the patient of the validation of the alarm, and this may allow the patient to know that he can prevent a fall alarm from being emitted in the case of a false fall by getting up as long as the first signal is still emitted.

The invention may moreover avoid the need for a patient to make efforts to get up in order to attempt to avoid the validation of the alarm although the second signal expressing validation thereof has already been emitted.

Furthermore, the fact of knowing through the emission of the second signal that the fall alarm has been validated may reassure the patient who has suffered the fall.

The subject of the invention is furthermore, independently or in combination with the foregoing, a fall impact sensor integrated into a multisensor terminal fixed to the belt of a patient, the multisensor terminal comprising a microcontroller designed to interpret and denoise the information coming from the fall impact sensor, the fall impact sensor comprising at least three arms, for example four arms, each arm comprising an inclination detector inclined with respect to the axis of the patient's trunk, for example positioned at 45° with respect to the axis of the patient's trunk, and an acceleration sensor positioned in a plane normal to the axis of the patient's trunk, the inclination detector being linked by one of its terminals to the acceleration sensor.

In all that follows, by “axis of the patient's trunk” is meant the axis which is vertical when the patient is standing upright.

The projections of the arms of the fall impact sensor in a plane normal to the axis of the trunk of the patient wearing the multisensor terminal may form, pairwise, angles of 90° when the fall impact sensor comprises four arms or angles of 120° when the fall impact sensor comprises three arms.

In each arm, one of the two electrical terminals of the acceleration sensor may be connected to an input in interruption of the microcontroller, the other electrical terminal being connected to a first electrical terminal of the inclination detector whose other electrical terminal is connected to ground, thereby allowing all the acceleration sensors to be connected to the same input in interruption of the microcontroller. The inclination detectors of each arm may moreover be directly connected to four distinct port inputs of the microcontroller.

Such a fall impact sensor exhibits for example very reduced or indeed zero electrical consumption, and makes it possible to detect a fall in the recumbent position of the person provided this impact is recorded in the direction of the fall, and this may constitute a preconditioning intrinsic to the fall sensor.

The microcontroller may execute an algorithm making it possible to discern the standing or seated position from the recumbent position, but also to detect falls of the patient when the latter passes from the standing or seated position to the recumbent position, or else during a fall from the recumbent position, when the patient is recumbent on a bed for example.

The algorithm may store in memory of the microcontroller the state of the inclination detectors for a predefined duration, for example three seconds, and this may make it possible to determine in the case of microcontroller interruption triggered by an impact on the floor whether the patient was standing or seated for at least a portion of the predefined duration preceding the interruption, for example for one second when the predefined duration is three seconds, or by default, whether the patient's trunk has pivoted by at least 180° during the predefined period preceding the interruption.

The microcontroller may be configured to emit a fall alarm to a local processing base if the patient remains recumbent during a predefined time interval, for example thirty seconds, subsequent to an impact on the floor taken into account despite any aborted attempts to get up. As a variant, the aforesaid time interval is for example ninety seconds.

The invention may be better understood on reading the detailed description which will follow, of an exemplary implementation thereof, and on examining the appended drawing, in which:

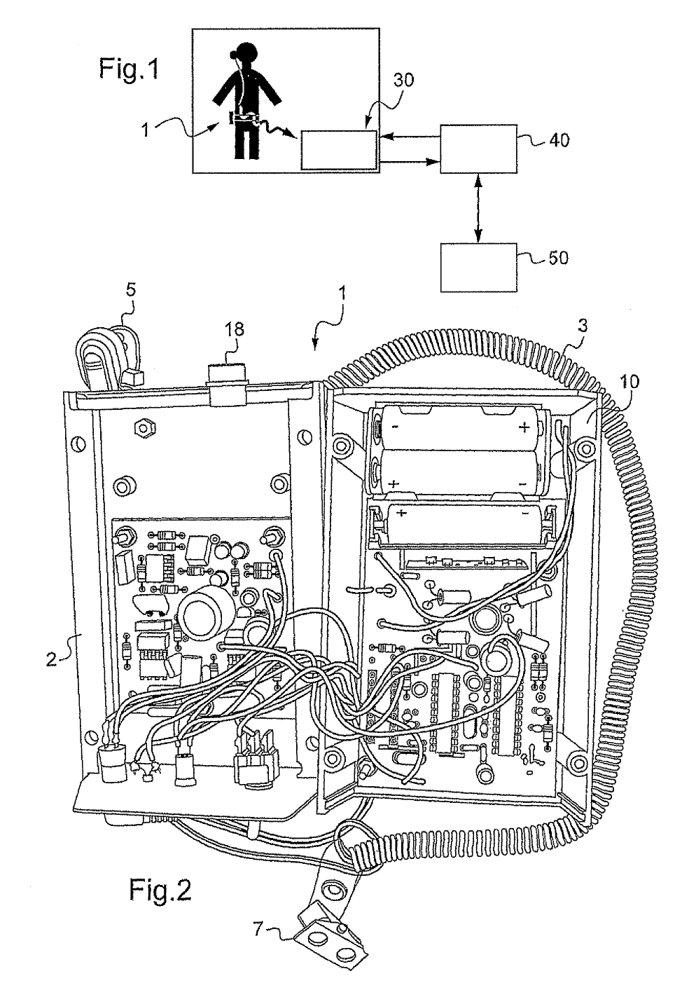

FIG. 1 represents an exemplary device according to the invention,

FIG. 2 represents in a schematic manner an exemplary multiplier terminal,

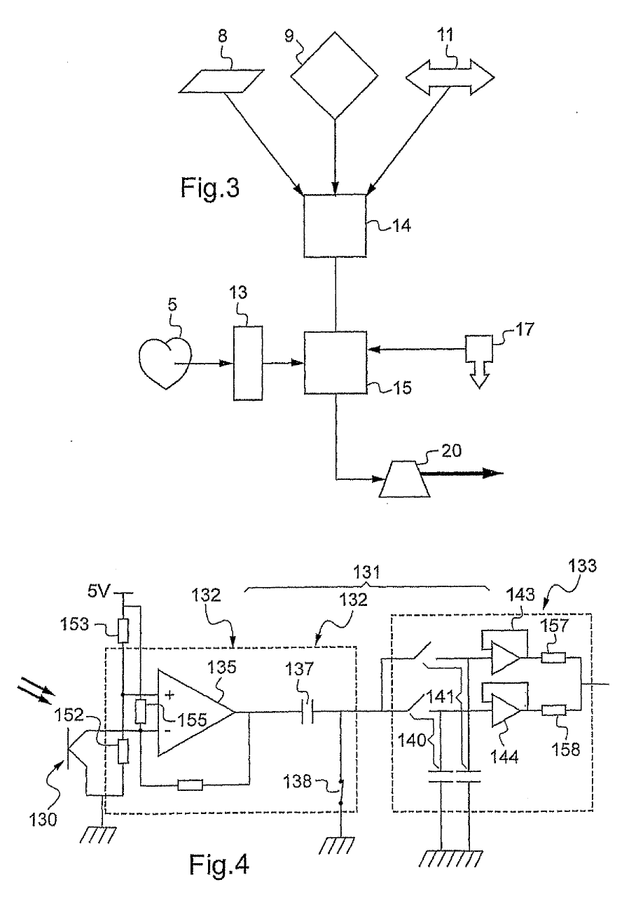

FIG. 3 is a block diagram of the device of FIG. 2,

FIGS. 4, 5 and 6 represent parts of the electronic preconditioning circuit in a schematic manner,

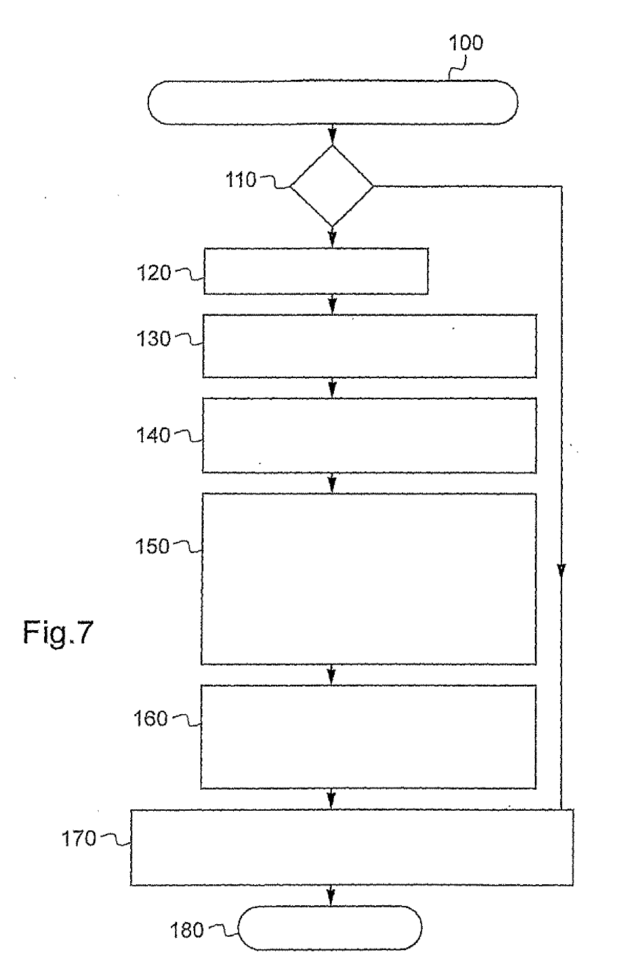

FIG. 7 represents, in schematic form, steps performed during the execution of the algorithm for eliminating the fast artifacts,

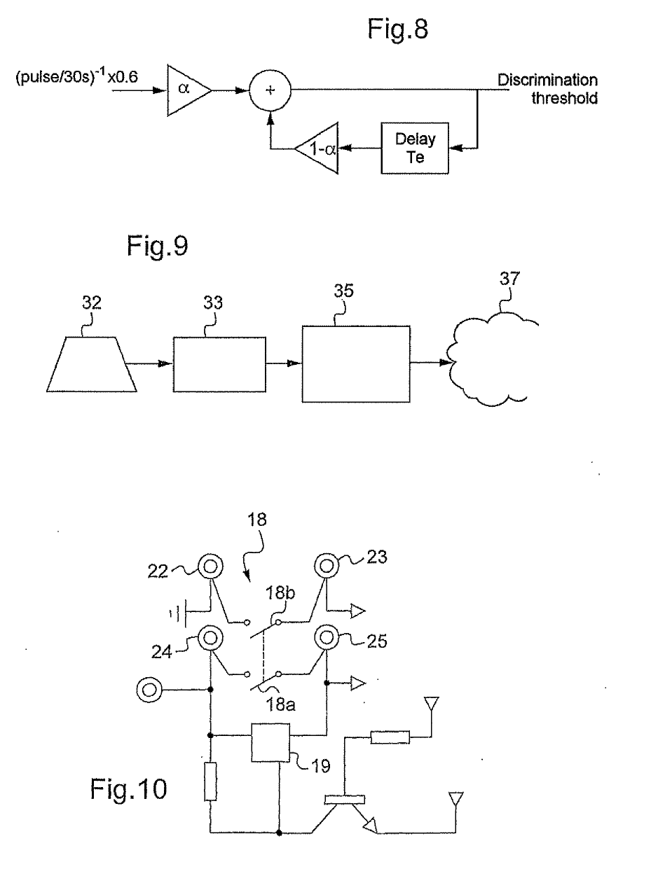

FIG. 8 represents a diagram of a method for recursively estimating the beat/artifact discrimination threshold according to the invention,

FIG. 9 represents in a schematic manner an exemplary local base,

FIG. 10 represents in a schematic manner the electronic circuit of the on/off switch, and

FIG. 11 represents an example of a fall impact sensor according to the invention.

An exemplary device in accordance with the invention has been represented in FIG. 1.

This device comprises a multisensor terminal 1 and a local base 30 that can communicate with a medical center 40 as well as with a treating doctor 50, if appropriate, or else with a predefined neighbor.

An exemplary embodiment of the multisensor terminal 1 has been represented in FIG. 2. This multisensor terminal comprises a box 2 fixed for example to the belt of the patient by way of a clip 7 or the like.

The terminal comprises a photophlethysmographic pulse sensor 5 connected to the box 2 by way of a cable 3. As a variant, this sensor 5 can communicate with the box 2 by way of a radio-frequency link.

The box 2 also comprises a housing 10 intended to receive cells for powering the system. As a variant, the power supply can be ensured by a rechargeable battery. A multisensor terminal 1 such as this can exhibit very reduced electrical consumption, of for example 3 mA.

The multisensor terminal 1, represented in a schematic manner in FIG. 3, can also comprise, as illustrated, an inclination sensor 8, a fall impact sensor 9, a movement sensor 11, two microcontrollers 14 and 15 as well as an emergency call button 17.

The box 2 further comprises a VHF emitter 20 whose radio range is between fifteen and twenty meters indoors and which emits at a frequency of 433 MHz, for example. As a variant, it may be a UHF emitter emitting at a frequency of 2.4 GHz or 868 MHz, for example.

In another variant, the radio link may be bidirectional.

The pulse sensor 5 used in the example described is an ear clip and may comprise one or more LEDs constituting a single light source and emitting an infrared beam, for example at 950 nm, and a light sensitive component which may comprise a phototransistor or, as a variant, one or more infrared photodiodes integrated into the same light sensitive component, allowing a measurement, by refraction through the ear lobe, of the attenuation of the infrared beam arising from the LED or LEDs.

The sensor 5 may also comprise an optical device adapting the geometry of the infrared beam.

The light sensitive component may comprise an infrared filter attenuating the disturbances due to the ambient light.

This sensor 5 may be provided with a hook disposed behind the ear or may, as a variant, be housed in the arms of a spectacle frame and may comprise one or more cells, be recharged in an inductive manner or comprise a photobattery.

The box 2 may comprise an on/off switch 18 which is of electromechanical and/or semi-conductor type. In an exemplary embodiment of the invention, the switch 18, represented in FIG. 10, comprises two breakers 18a and 18b controlled together mechanically. The switch 18 also comprises an electronic breaker 19 which in the example described is a field-effect transistor of MOS type, arranged so as to shunt the breaker 18a.

The switch 18 may not be soldered on the electronic card of the multisensor terminal 1 and be for example connected thereto by flexible wires from connection points 22, 23, 24 and 25.

The inclination sensor 8 comprises four sensors preferably based on drops of mercury or other conducting liquid. Such sensors are less disturbed by the movements of the patient than ball-type sensors. The inclination sensors 8 are distributed in four directions which are pairwise orthogonal, the directions corresponding to the generators of a downward pointing cone, open at 90° and whose axis coincides with that of the patient's trunk.

The movement sensor 11 comprises a ball-type sensor, for example with the ASSEMtech brand, connected to an input of the microcontroller 14. This movement sensor, after conditioning by the microcontroller 14, makes it possible to total up the number of seconds for which the patient exhibits the least physical activity and may make it possible to ascertain the temporal rate of movement of the patient.

The fall impact sensor 9 is for example constructed around a composite sensor providing the indication regarding position (recumbent or standing/seated) and fall impact on the floor. This sensor comprises for example four arms an example of which has been represented in FIG. 11. Each arm 90 comprises a detector of inclination 91 with respect to the horizontal axis, for example similar to the inclination sensor 8, and an acceleration sensor 92, for example a breaker detecting overshoot of an acceleration threshold fixed for example at 2 g.

The inclination detectors 91 are in the example described positioned at 45° with respect to the axis X of the trunk of the patient wearing the multisensor terminal 1 and the acceleration sensors 92 may be positioned in a plane Y normal to the axis X of the patient's trunk. The breakers detecting overshoot of the acceleration threshold 92 are for example designed to briefly close when the projection of the acceleration on their axis overshoots the fixed acceleration threshold.

In each arm 90, the inclination detector 91 comprises two electrical terminals 93 and 94. The first electrical terminal 93 is connected to ground, the second electrical terminal 94 is connected to a first electrical terminal 96 of the acceleration sensor 92 of the same arm. The second electrical terminal 97 of the acceleration sensors of each arm is connected to the same input in interruption of the microcontroller 14. Moreover, the inclination detector 91 of each arm 90 is connected directly by its second electrical terminal 94 to distinct port inputs of the microcontroller 14.

An impact sensor with four orthogonal arms proceeding by orientation filtering is thus obtained in this example, each of these arms being composed of an inclination detector and of an acceleration sensor in series. One speaks of orientation filtering since this device records a fall impact only if an impact is detected in the recumbent position in the direction where the patient is recumbent.

The microcontroller 14 is designed to interpret and denoise the indications coming from the inclination sensor 8 and movement sensor 11.

The microcontroller 14 is designed to process the indications received from the fall impact sensor 9, to associate a value with a variable when a fall indication is detected, and thereafter to emit a fall alarm if the variable retains the same value for a predefined duration, lying preferably between ten and a hundred seconds, for example thirty seconds, that is to say if the equipped person remains continually recumbent for this duration.

The microcontroller 14 is for example designed to discern the standing or seated position from the recumbent position as well as to detect any falls of the patient, either from a seated or standing position to a recumbent position or during a fall from a recumbent position, this being for example the case when a patient falls out of the bed in which he is recumbent.

In the example described, the microcontroller 14 is configured to store in its memory the state of the inclination detectors for a predefined duration, being equal to three seconds in the example described, so as to determine in the case of an interruption of the microcontroller 14 triggered by an impact on the floor whether, for the predefined duration preceding the interruption, the patient was standing or seated or whether the trunk of the patient in the recumbent position has pivoted by more than 180°.

In an exemplary embodiment of the invention, when a patient who has suffered a fall tries vainly to get up, the measurements acquired after the fall which no longer express a recumbent position ought not modify the value of the variable associated with the fall indication. Such detection by the microcontroller 14 of the return to the standing/seated position may be insensitive to any vain attempts by the patient to get up.

With this aim, if a fall impact has been detected by the fall impact sensor 9, the microcontroller 14 may be designed so as, within the framework of the validation of the fall alarm, to modify the frequency of sampling of the measurements of the actimetry sensors, especially of the inclination sensors. The microcontroller 14 can sample for example every 50 ms the state of the inclination sensor 8 and detect the standing/seated state for a fraction, for example ⅓, of the samples acquired over the predefined duration of validation of the sending of the fall alarm. When the predefined duration of validation of the fall alarm is thirty seconds, the microcontroller 14 can detect the standing/seated state every ten seconds for three seconds.

The value of the variable associated with the fall indication may not be modified, and this may lead to the sending of the fall alarm on completion of the predefined duration.

In another exemplary implementation of the invention, the duration of validation before emission of the alarm may be higher.

In another exemplary implementation of the invention, the multisensor terminal 1 comprises a buzzer emitting a first audible signal lasting for a duration of about 100 ms every three seconds as long as the fall alarm has not been validated and then a second signal lasting for a duration of about 1 second when the alarm has been validated.

The fall alarm is emitted as soon as it has been validated at the local base 30 and can then be re-emitted during a following ordinary emission of data.

The microcontroller 14 sends the denoised indications to the microcontroller 15 periodically, for example every fifteen seconds.

In the example considered, the microcontroller 15 sends a signal on several octets to the emitter 20 for example every thirty seconds. The first octet comprises for example indications relating to the actimetry measured by the inclination sensor 8, fall impact sensor 9 and movement sensor 11. These indications can relate to the charge level of the battery or of the cells of the box 2 or else to a possible action on the emergency call button 17. The second octet corresponds for example to a label, the third octet comprises for example pulse related indications arising from the denoising method and the fourth octet corresponds for example to the repetition of the previous label, and this may provide a means of discerning a transmission error in the data received and of enhancing the reliability, in the case of use in an old people's home or in a care center, of the knowledge of the origin of the data for a use of several multisensor terminals 1 with a single local base 30.

The microcontroller 15 is also configured in the example considered to supervise the operation of the emergency call button 17 whose actuation may trigger the immediate sending of the information of the sensors not older than for example fifteen or thirty seconds and the automatic repetition of this emission in the following thirty seconds, for example.

The microcontroller 15 may be configured to supervise the sensor 5 and the preconditioning circuit 13 which will be described subsequently. The microcontroller 15 is for example configured to process the signal delivered by the preconditioning circuit 13, and this may make it possible to attenuate relatively strongly the errors due to the patient's movement artifacts in the measurements sent to the local base 30 for receiving the number of beats during a time interval equal for example to thirty seconds.

The supervision mentioned above may, as a variant, be performed by the microcontroller 14.

When the patient decides to cut off the power supply to the box 2, he can exert an action on the switch 18 so as to place the latter in the “off” position.

This action gives rise to the opening of the breaker 18b and the sending to the microcontroller 15 of an indication relating to this opening of the breaker 18b.

The microcontroller 15 may be configured to keep the MOS transistor 19, used as breaker and placed in parallel with the breaker 18a, passing and to cause the immediate sending of the current indications to the local base 30.

The microcontroller 15 may keep the MOS transistor 19 passing as long as the indications transmitted periodically to the local base 30 have not been sent. When the periodic sending of current indications to the local base 30 has been performed, the microcontroller 15 may cut off the power supply to the box by acting on the gate of the MOS transistor 19.

The immediate sending to the local base of indications and the sending of periodic indications may allow the local base to verify the likelihood of the immediately sent indications, and this may constitute a cue relating to the reliability of these indications.

In a variant, the microcontroller 15 may be designed to cut off the power supply by acting on the breaker 19 right from the immediate sending of indications to the local base 30.

An exemplary embodiment of the local base 30 is represented in FIG. 9. The local base 30 receives the information sent by the terminal 1, in particular by the emitter 20. Accordingly it comprises a receiver 32, for example a VHF receiver of frequency for example equal to 433 MHz or a UHF receiver, for example at 2.4 GHz or 868 MHz. It also comprises a microcontroller 33 managing the reception of the information, a computer or computerized system 35 and a link, for example a RS 232 serial link, linking the microcontroller 33 to the serial port of the computer 35. As a variant, the microcontroller 33 and the computer 35 could be connected by a parallel link or a USB link, for example.

The local base 30 is connected by a network 37, possibly the Internet or any other wired network or else an non-wired network, with a centralized telemonitoring server situated in the medical center 40. A duty doctor 50 and/or a nurse of the medical center 40 may emit a prediagnosis on the basis of the indications that have been transmitted to them from the local base 30 by the network 37 and of the patient's personal data stored in a database of the medical center 40.

The computer 35 is, in the example described, configured to process the information arising from the sensors, merge them with the aid of decision rules and record these information in a daily file and then an archive file; an archiving base whose depth or number of days is adapted to the utilization of the system, for example thirty days, can for example be produced. The computer 35 comprises, in the example described, a screen making it possible to display the data thus processed. The PC 35 is also configured to add two pulse values calculated over thirty seconds, for example the n^{th} pulse value, transmitted by the microcontroller 15 of the multisensor terminal 1 to the n−1^{th} pulse value so as to provide a value in number of beats by minute. If appropriate, the local base 30 can emit a fall alarm, or else an alarm corresponding to the patient's action on the emergency call button 17.

The alarms may be transmitted to persons selected by the patient such as a parent, a friend or a neighbor.

In response to a removing of the sensor 5 by the patient, the local base 30 may be configured to substitute an alarm value for the pulse value, and to cause the display on the screen of the computer 35 and the transmission to the medical center 40 of a warning message, for example “clip removed”. This alarm value may be chosen so as not to be able to be interpreted as a plausible beat value. The maximum pulse frequency accepted for the targeted population being 100 beats per interval of thirty seconds, the chosen value will be for example greater than 110.

The multisensor terminal 1 may be configured to detect whether the clip of the sensor 5 is disconnected from the box 2 by the patient or whether the power supply wire for the LEDs is cut inside the box 2, or else in the cable 3 of the pulse sensor, or else if the plug of the cable 3 is incorrectly engaged in a socket of the corresponding box, so as to substitute an alarm value chosen for example as above for the value of the pulse and to cause the display on the screen of the computer 35 and the transmission to the medical center 40 of a warning message, for example “clip disconnected”.

The device may for example make it possible to inform the medical center 40 of the state of use of the box and of any faults with the multisensor terminal so as to avoid, if appropriate, the unnecessary and deleterious sending of a medical vehicle.

The centralized telemonitoring server situated in the medical center 40 may be configured to program the local base 30 or the multisensor terminal 1. The program of the centralized server may, as already mentioned, make it possible to determine whether or not the pulse sensor 5 clip is being worn, whether the clip of the pulse sensor 5 is disconnected from the multisensor terminal, whether or not the charge state of the battery or of the cells of the power supply to the box is sufficient, or else whether the multisensor terminal has been turned off intentionally.

The program of the centralized server may also be configured to check the operation of the local base 30 from the computer 35 by returning a specific message while testing for example the normal operation of the microcontroller 33 by sending a reinitialization order (reset of the microcontroller) to this microcontroller 33, triggering in return the sending of a specific message to the computer 35.

The denoising device previously mentioned, implemented in the electronic preconditioning circuit 13, and the microcontroller 14 for processing the indications coming from the sensor 5 will now be described.

This sensor 5 comprises in the example described three LEDs emitting an infrared beam through the ear lobe of a user and constituting a single light source. With the aim of limiting the consumption of the LEDs, the latter are for example supplied by way of the microcontroller 14 or of a microcontroller housed in the sensor 5 which delivers a rectangular current of low duty ratio, preferably less than 1/10, lying for example between 1/40 and 1/10, for example equal to 1/20.

The infrared beam illuminates a light sensitive component, in the example described a phototransistor 130. The power supply to the LEDs is for example such that they are lit for a time equal to 500 μs.

The invention is of course not limited to the employing of a phototransistor as light sensitive component. In another exemplary implementation of the invention, the light sensitive component is a photodiode and the power supply to the LEDs is such that they are lit for a time equal to 50 μs.

The LEDs power supply frequency may be chosen to satisfy the constraints relating to the autonomy of the multisensor terminal 1 and the elimination of frequencies modulating the sources of artificial light which result generally from the mains frequency, i.e. 50 Hz and its harmonics for example. The power supply frequency for the LEDs may advantageously be equal to 200 Hz.

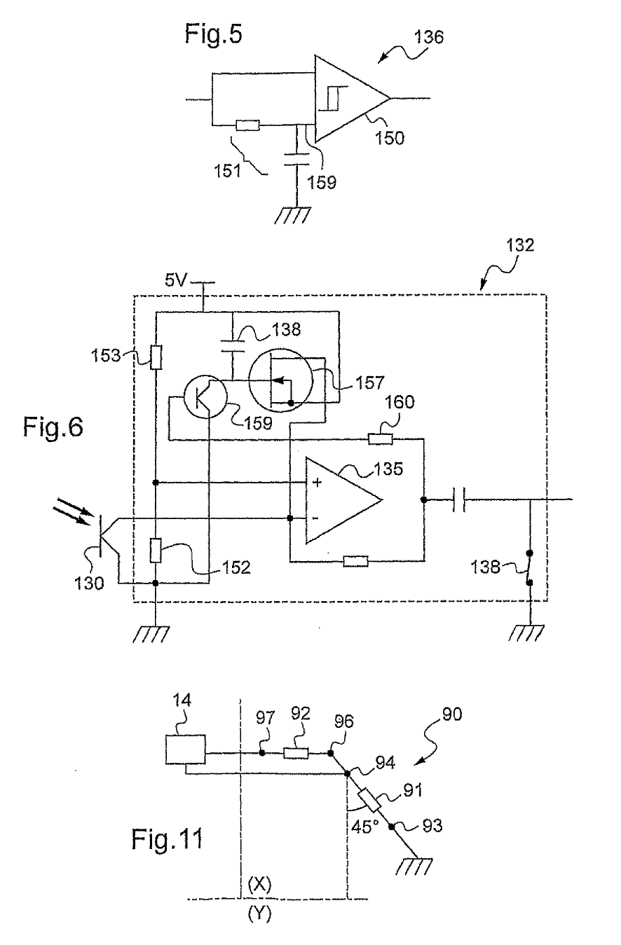

The signal arising from the sensor 5 thereafter drives the preconditioning circuit 13, which is configured to eliminate the slow artifacts present in the signal of the pulse sensed by the sensor 5. The preconditioning circuit 13 is partially represented in FIGS. 4 and 5.

This preconditioning circuit comprises in the example described a filtering chain 131 comprising a stage 132 for subtracting additive noise, a stage 133 for filtering the frequencies which are multiples of the fundamental of the electrical power supply, a high-pass filter, a variable gain, and a low-pass filter.

The preconditioning circuit 13 further comprises a stage 136 termed a “floating trigger” consisting of a voltage comparator with hysteresis.

The signal sensed by the phototransistor 130 and corresponding to the illumination of the patient's lobe drives the circuit 13 by way of the additive noise subtraction stage 132.

This stage 132 is configured to subtract the components of the noise with slow variations, for example caused by a change of position of the patient's head with respect to a main source of ambient light. Nuisance noise such as this generally constitutes an obstacle to the use of multisensor terminals while ambulatory, being engendered by the least movement.

The signal arising from the phototransistor 130 drives the input—an operational amplifier (O.A.) 135. The + input of the O.A. 135 is supplied by a voltage divider bridge comprising two resistors 152 and 153 and whose supply voltage is for example 5 V. By choosing resistors 152 and 153 of the same value and by supplying the system with a voltage of 5 V, the + input of the O.A. is at a potential of 2.5 V and the output voltage of the O.A. varies between 2.5 and 5 V. In order to obtain a variation of the output voltage of the O.A. 135 of between 0 and 5 V, a resistor 155 is arranged in series between the power supply (for example 5V) and the − input of the O.A. 135. The signal at the output of the O.A. 135 thereafter drives an assembly comprising a capacitor 137 and an electronic breaker 138, for example a field-effect transistor or an analog breaker, for example marketed by the company MAXIM®.

When the phototransistor 130 is illuminated, the breaker 138 is open. The voltage measured across the terminals of the capacitor 137 corresponds in this case to the illumination arising from the infrared LEDs and that has for example passed through the ear lobe to which is added the noise (slow variations of the ambient light, etc.) independent of the lighting arising from the LEDs.

When the infrared LEDs of the sensor 5 are unlit, the breaker 138 is closed. The voltage measured across the terminals of the capacitor corresponds in this case to the noise independent of the illumination by the LEDs.

The lit-unlit cycle of the LEDs makes it possible to subtract the additive part of the slow variations of the illumination at the level of the phototransistor, which are due to the movement of the patient's head as well as to the diverse modifications of the illumination of his close environment, the capacitor 137 having across its electrical terminals a voltage representative of this additive part when the phototransistor 130 is not illuminated by the LEDs.

The stage 133 makes it possible to eliminate from the frequency spectrum the multiples of the fundamental of the electrical power supply.

The ambient lighting is generally supplied at 50 Hz, thus corresponding to a nuisance signal comprising a fundamental at 100 Hz as well as several harmonics, especially at frequencies which are multiples of 100 Hz. In the case where the frequency of the electrical power supply is 60 Hz, the nuisance signal comprises a fundamental at 120 Hz and harmonics at multiples of 120 Hz.

The stage 133 is configured to perform a discrete-time filtering of the signal arising from the stage 132, without there being digitization of this signal. The complexity of the device is thus reduced.

This stage 133 comprises two acquisition pathways. Each pathway comprises a sample-and-hold unit 140 or 141 arranged in series with a so-called follower arrangement consisting of an O.A. 143 or 144. One does not depart from the framework of the present invention when the stage 133 comprises only one acquisition pathway and only one sample-and-hold unit.

The outputs of these O.A. 143 and 144 are respectively connected to the resistors 157 and 158.

In the example described, these two resistors 157 and 158 are of the same value with a tolerance for example equal to 0.1%.

A summation of the signals originating from each sample-and-hold unit 140 and 141 is performed at the output of the stage 133.

The sample-and-hold units 140 and 141 are controlled alternately, in such a way that the signal travels through one pathway or through the other, with no possibility of overlap.

During the phases when the LEDs are unlit, the phototransistor 130 is not illuminated. The sample-and-hold units 140 and 141, which in the example described are hold units of order zero allow, when the LEDs are unlit, the maintenance of the signal recorded during the illumination of the phototransistor by the LEDs.

The control by alternation of the sample-and-hold units 140 and 141 makes it possible for the n^{th} measurement to be acquired by one pathway alone, the (n−1)^{th} having been acquired by the other.

The stage 133 performs two filtering operations in cascade.

The first operation is a sampled-time filtering with recurrence equation

$$\frac{x ( n ) + x ( n - 1 )}{2}$$

whose frequency response, of the comb filter type, is

$$cos ( \frac{ω T}{2} )$$

where ω is the angular frequency of the signal and T the sampling period. This response vanishes at odd half-multiples of the sampling frequency.

The second operation is a continuous-time filtering carried out by the “hold unit of order zero” function whose frequency response is

sin c(πfT).

This response constitutes a low-pass filtering whose frequency response vanishes at multiples of the sampling frequency.

The cascading of these two filtering operations may make it possible to obtain a filtering devoid of group propagation time distortion or else with constant group propagation, and this may make it possible to best retransmit the shape of the useful signal before it is shaped by the floating trigger stage 136.

The stage 133 being configured in the example considered to allow the elimination from the frequency spectrum of the multiples of 100 Hz. The sampling frequency is chosen equal to 200 Hz, thereby allowing control alternating at 100 Hz of the sample-and-hold units 140 and 141.

In the case where the frequency of the electrical power supply is 60 Hz, the sampling frequency is chosen equal to 240 Hz.

The signal at the output of the stage 133 is devoid of noise whose frequency is a multiple of the fundamental (100 or 120 Hz) of the mains powered electrical lighting.

The preconditioning circuit 13 comprises furthermore, in the example described, a high-pass filter of cutoff frequency 0.5 Hz, a variable gain amplifier and a low-pass filter of cutoff frequency 10 Hz. The signal arising from these filters is a signal smoothed in a spectral window extending for example between 0.5 Hz and 20 Hz, or else between 0.5 Hz and 10 Hz. The reduction of the spectral window containing the signal may make it possible for example to increase the value of the signal/noise ratio insofar as the fast transitions of the signal (systolic edges) are correctly retransmitted by this bandpass filtering so as not to disturb the operation of the “floating trigger” stage 136. By “correctly retransmitted” is understood to mean with low distortion of amplitude and of group propagation time.

The signal arising from the stage 133 comprises the fast edges arising from the sensor 5 but the high-frequency noise has been removed.

In order to obtain a rectangular signal at the output of the preconditioning circuit 13, the signal drives the so-called “floating trigger” stage 136 represented in FIG. 5.

This stage 136 comprises a voltage comparator with hysteresis 150 which compares the signal arising from the filtering chain with a signal at the output of a filter 151. This filter 151 is of low-pass RC type used as approximately constant delay element and presents at its output 159 a slightly delayed version of the input signal.

The signal arising from this comparator 136 is a logic signal exhibiting a falling edge on each pulse beat but also in the presence of artifacts of fast movements whose amplitude exceeds the threshold of the comparator 136, such as for example a bang on the sensor or a fast movement of the head.

Another exemplary stage 132 for subtracting additive noise has been represented in FIG. 6. In this example, the stage 132 differs from that described with reference to FIG. 4 by the replacement of the resistor 155 by a P-channel MOSFET transistor 157. The source of this transistor 157 is supplied by the supply voltage which in the example described is 5V. The drain of the transistor is connected to the − input of the O.A. 135. A capacitor 138, with capacitance of for example between 2 nF and 10 nF, is connected between the gate and the source of the transistor 157. The capacitor 138 is in the example described designed to store the control voltage corresponding to the provision of a current substantially equal but opposite to the photo-current due to the ambient lighting, and this may make it possible to increase the preconditioning circuit 13 ambient light additive noise rejection dynamics.

The gate of the transistor 157 is in the example described connected to the collector of an NPN bipolar transistor 159 whose base is connected by a resistor 160, for example a resistor of between 5Ω and 10 kΩ, to the output of the O.A. 135. The emitter of the bipolar transistor 159 is for example connected to ground. The stage 132 can further comprise an electronic breaker (not represented), for example a field-effect transistor, for interrupting the control circuit of the transistor 157 when the LED or LEDs of the sensor 5 are lit, the capacitor 159 then storing the voltage corresponding to the ambient light photocurrent to be compensated.

Another analog breaker, for example another P-channel MOS transistor shunting the capacitor 159, may allow its discharging just after the extinguishing of the infrared LEDs before its recharging for the duration of extinguishing of these LEDs at the voltage corresponding to the compensation of the ambient light photocurrent.

The microcontroller 15 executes an algorithm designed to eliminate the fast artifacts contained in the signal arising from the electronic preconditioning circuit 13.

The basic principle of this algorithm is the elimination of the supernumerary falling edges due to the artifacts of fast movements present in the logic signal arising from the floating trigger stage 136, by measuring the inter-beat interval and adapting the beat/artifact discrimination threshold.

An exemplary algorithm for denoising fast artifacts, executed by the microcontroller 15, will now be described in greater detail, this algorithm being designed to reckon up the number of beats emitted per interval of thirty seconds.

The denoising method represented in FIG. 7 makes it possible to improve the heuristic relying on the measurement of the inter-beat interval by virtue of a blanket removal of the edge effect, that is to say the subtraction of a number of beats which are independent of the duration of the noise-affected zone, at the ends of the zones undergoing an uninterrupted disturbance by fast artifacts detected by the algorithm, so as to center the residual error.

This FIG. 7 represents an example of the interruption subroutine executed by the microcontroller 15. This subroutine is invoked periodically, for example every fifteen seconds, by the microcontroller 14 managing the data arising from the actimetry sensors 8, 9 and 11.

During a first step 100, data are transmitted by a link, for example a serial link, originating from the microcontroller 14 to the microcontroller 15 every fifteen seconds.

During a step 110, the microcontroller 15 evaluates whether an emergency call signal originating from the call button 17 has been emitted. When this is the case, the microcontroller directly executes a step 170 corresponding to the setting to “1” of the flag triggering the radiofrequency sending of data by the emitter 20 to the local base 30.

When no emergency call has been emitted, the microcontroller increments, in the course of a step 120, the beat counter or the value of the current noise-affected time.

Subsequent to this incrementation step, in the course of a step 130, a blanket subtraction of for example a half-beat per noise-affected zone is performed, a zone affected by noise in an uninterrupted manner corresponding to a disturbance. This subtraction makes it possible to take the edge effect into account and to center the residual measurement error. In the course of this step, the pulse value is corrected a first time according to the equation:

$$pulse ( n ) = pulse ( n ) - \frac{number of noise affected zones}{2}$$

In the course of a step 140, the microcontroller 15 performs an addition, prorata temporis of the noise-affected time, of a number of beats corresponding to the pulse corrected at the previous instant and available as output of the algorithm. This step may make it possible to fill in the aggregated duration of the noise-affected zones during the current counting period, for example fifteen seconds, according to the following relation:

pulse(n)=pulse(n)+noise-affected time×pulse(n−1)

In the course of step 150, the pulse is corrected again. In the course of this step 150, the last two intervals (n) and (n−1) are considered. Several cases may then arise:
if the last two intervals of fifteen seconds each exhibit a temporal noising rate of greater than 17%, the algorithm causes the sending of a denoising failure alarm and effects a reduction in the beat/artifact discrimination temporal threshold, it not being possible for the new threshold to be less than the threshold initially fixed at 0.3 seconds. Here one speaks of failure of the denoising.
if only one of the last two intervals of fifteen seconds exhibits a temporal noising rate of greater than 17%, that is to say if

noise-affected time(n)>17%×15 s or noise-affected time(n−1)>17%×15 s

then the pulse over 30 seconds is calculated over the interval of fifteen seconds not exhibiting more than 17% of noise-affected time,
if the temporal noising rate during the last thirty seconds is less than 17%, and greater than 5%, the algorithm calculates a mean over the last two intervals of fifteen seconds, weighted by the noise-affected durations of each interval according to the following relation:

$$pulse / 30 s = pulse ( n ) + pulse ( n - 1 ) + [ pulse ( n - 1 ) - pulse ( n ) ] \times \frac{\begin{matrix}noise affected time ( n ) - \\ noise affected time ( n - 1 )\end{matrix}}{\begin{matrix}noise affected time ( n ) + \\ noise affected time ( n - 1 )\end{matrix}}$$

if the temporal noising rate during the last thirty seconds is less than 5%, the algorithm calculates the pulse over these thirty seconds according to the following relation:

pulse/30 s=pulse(n)+pulse(n−1)

Step 160 corresponds to a reduction in the beat/artifact discrimination threshold.

Two cases may then arise:
in the case of failure of the denoising in step 150, that is to say when the last two intervals of fifteen seconds each exhibit a temporal noising rate of greater than 17%, the new discriminating threshold is chosen as being equal to a fraction, for example to three quarters, of the previous discrimination threshold,
in the other cases, a recursive estimator of the discrimination threshold is used. This recursive estimator allows a noticeable improvement in the removal of the edges of fast artifacts by adapting the initial beat/artifact discrimination threshold of 0.3 seconds to the patient's mean pulse.

A convergence index for this estimator thereafter makes it possible to suppress the heuristic removal of the edge effect performed in step 130 which is no longer needed if the estimator converges, that is to say if the patient's pulse does not deviate substantially from his mean pulse.

FIG. 8 represents an exemplary method for recursively estimating the beat/artifact discrimination threshold. The input for this method is the inverse of the pulse over the last thirty seconds, multiplied by a factor of 0.6 to ensure the detection of any artifact placed between two consecutive real beats while affording a possibility of an increase in the cardiac frequency of the patient up to for example 1.67 times the current mean pulse. The resulting value is thereafter multiplied by a constant α much less than 1. This new value is thereafter added to the previous discrimination threshold delayed by the sampling period and multiplied by (1−α). α is for example between 1/100 and 1/2. In the example described, α is equal to 1/30, thus yielding a duration of convergence of the recursive estimator of between 30 min and 45 min.

If the beats/artifact discrimination threshold formulated by the recursive estimator represented in FIG. 8 is greater than or equal to half the mean interbeat calculated over the last thirty seconds, the blanket removal of the edge effect performed in step 130 is no longer needed, any beat resulting from an artifact encroaching between two consecutive real beats then being either detected by the algorithm as an artifact and does not increment the counter of real beats in step 120, or detected as a real beat, thereby giving rise to the false interpretation of the following real beat as an artifact by the algorithm, these two cases having the same bearing on the aggregate of the real beats during an interval of fifteen seconds.

Subsequent to this method for recursively estimating the beat/artifact discrimination threshold, the microcontroller 15 executes step 170 which triggers the radiofrequency sending by the emitter 20 of indications to the local base 30, such as for example the number of beats in the course of the last thirty seconds.

Step 180 constitutes an interruption return.

The microcontroller 33 which manages the receiver of the local base 30 may check by various tests the integrity of the signal received so as to prevent radio signals not intended for the domestic base from being interpreted by the latter as indications arising from the multisensor terminal 1.

The invention is not limited to the implementation examples just described.

The multisensor terminal may for example be coupled to a home-automation device for monitoring the patient, such as for example infrared sensors distributed in various places in the rooms of the patient's residence and allowing location of the patient in his residence, or for example further be coupled to an actimetric floor locating the patient in his residence.

As a variant, the home-automation device for monitoring the patient to which the multisensor terminal may be coupled may comprise a network of receivers/emitters according to the Zigbee standard making it possible to locate the patient while securing the links between his multisensor box and the local base, the emitters/receivers communicating for example with the local base by carrier currents.

The expression “comprising a” should be understood as being synonymous with “comprising at least one”.

1. A device for measuring the pulse comprising:
a pulse sensor of photoplethysmographic type comprising at least one light source, especially an infrared light source, and a component sensitive to the light emitted by the source, especially a single component, and
a denoising system comprising:
an electronic preconditioning circuit configured to eliminate the slow artifacts of a signal representative of the pulse acquired by the light sensitive component and,
at least one microcontroller configured to process the signal delivered by the electronic preconditioning circuit and eliminate the fast artifacts.

2. The device as claimed in claim 1, the light source being supplied by a discontinuous current with duty ratio of less than 1/10, especially of between 1/40 and 1/10.

3. The device as claimed in claim 1, the electronic preconditioning circuit comprising a filtering chain for the sensed signal.

4. The device as claimed in claim 1, the filtering chain comprising a stage for subtracting additive noise.

5. The device as claimed in claim 3, the filtering chain comprising a stage for filtering the frequencies which are multiples of the fundamental of the electrical power supply of the light source.

6. The device as claimed in claim 5, the filtering stage comprising two sample-and-hold units controlled alternately.

7. The device as claimed in claim 3, the filtering chain comprising a filter cutting off the frequencies outside a spectral window of between 0.5 Hz and 20 Hz, especially between 0.5 Hz and 10 Hz.

8. The device as claimed in claim 3, the electronic preconditioning circuit comprising a voltage comparator with hysteresis comparing a signal arising from the filtering chain and a delayed version of this same signal.

9. The device as claimed in claim 8, the delay being between 20 ms and 40 ms.

10. The device as claimed in claim 1, the electronic preconditioning circuit being devoid of analog-digital converter.

11. A mobile monitoring system for a patient, comprising:
a multisensor terminal comprising a box to be worn by the patient, the terminal including the device for measuring the pulse as claimed in claim 1 and,
a local processing base for receiving and processing information sent, especially according to a predefined period, by the multisensor terminal.

12. The system as claimed in claim 11, the multisensor terminal comprising means for fixing to a belt.

13. The system as claimed in claim 11, the multisensor terminal comprising at least one actimetry sensor chosen from among: an isotropic movement sensor, an inclination sensor and a fall impact sensor.

14. The system as claimed in claim 13, the multisensor terminal comprising the fall impact sensor,
comprising four arms, each arm comprising in series an inclination sensor and an acceleration sensor.

15. A method for measuring the pulse of a person by means of a device comprising a pulse sensor comprising at least one light source and a component sensitive to the light emitted by the source, especially a single component, and a denoising system comprising:
an electronic preconditioning circuit configured to eliminate the slow artifacts of a signal representative of the pulse acquired by the light sensitive component, and
at least one microcontroller configured to process the signal delivered by the electronic preconditioning circuit and eliminate the fast artifacts.

16. The method as claimed in claim 15, comprising a step of preprocessing by the electronic preconditioning circuit in the course of which,
the signal arising from the pulse sensor drives a stage for subtracting the additive components of the noise with slow variations contained in the signal,
the signal is processed by a low-pass filtering stage with constant group propagation time and with removal of the frequencies which are multiples of the fundamental of the electrical power supply, and
the signal is put into logic form by means of a comparator stage.

17. The method as claimed in claim 15, comprising a step of algorithmic post-processing by the microcontroller in the course of which:
a removal of the edge effect is performed by subtracting from the number of beats measured per time interval a predefined number of beats per noise-affected zone, especially a half-beat per noise-affected zone,
the number of beats corrected during the previous time interval prorata-temporis of the noise-affected time is added to the value obtained,
the temporal noising rate is compared with a reference value, and the beat/artifact discrimination threshold is adapted.

18. The method as claimed in claim 17, comprising a recursive step of estimating the beat/artifact discrimination threshold.

19. The method as claimed in claim 17, the reference value of the temporal noising rate being between 10 and 30%.

20. A fall impact sensor integrated into a multisensor terminal fixed to the belt of a patient, the multisensor terminal comprising a microcontroller configured to interpret and denoise the information coming from the fall impact sensor, the fall impact sensor comprising at least three arms each comprising an inclination detector inclined ° with respect to the axis of the patient's trunk and an acceleration sensor positioned in a plane normal to the axis of the patient's trunk, the inclination detector being linked by one of its electrical terminals to the acceleration sensor.

21. The fall impact sensor as claimed in claim 20, comprising four arms and being configured so that the projections of the arms of the fall impact sensor in a plane normal to the axis of the trunk of the patient wearing the multisensor terminal form, pairwise, angles of 90°.

22. The fall impact sensor as claimed in claim 20, the acceleration sensor of each arm comprising two electrical terminals, one electrical terminal being connected to an input in interruption of the microcontroller, the other electrical terminal being connected to a terminal of the inclination detector.

23. The fall impact sensor as claimed in claim 22, the inclination detector of each arm comprising an electrical terminal connected to ground.

24. The fall impact sensor as claimed in claim 23, the inclination detectors being connected directly to four distinct port inputs of the microcontroller.

25. A method for detecting the fall of a person by means of a device comprising:
a fall sensor,
at least one microcontroller configured to process the signal delivered by the fall sensor, and
a component emitting with predefined periodicity a first signal audible by the patient as long as the fall alarm has not been validated and then, when the alarm has been validated a second signal audible by the patient, different from the first.
